(19) United States
(12) Patent Application Publication
(10) Pub. No.: US 2022/0031825 A1
(43) Pub. Date: Feb. 3, 2022

(54) IMMUNOLOGICAL ADJUVANT AND VACCINE COMPOSITION INCLUDING STING AGONIST

(71) Applicant: Quratis Inc., Seoul (KR)

(72) Inventors: Sung Jae Shin, Seoul (KR); Myung Ryurl Oh, Gyeonggi-do (KR)

(21) Appl. No.: 17/277,138

(22) PCT Filed: Sep. 17, 2018
(86) PCT No.: PCT/KR2018/010898

§ 371 Date: Mar. 17, 2021

(51) Int. Cl. A61K 39/04; A61K 39/39; A61P 31/06

(52) U.S. Cl. CPC A61K 39/04; A61K 2039/55561; A61P 31/06; A61K 39/39

(57) ABSTRACT

The present invention relates to an immunological adjuvant composition and a vaccine composition including a STING immunological adjuvant, and it is confirmed that the immunological adjuvant composition and vaccine composition including the STING agonist according to the present invention not only may effectively activate various body immune responses, but also have the effect of remarkably reducing the infection of Mycobacterium tuberculosis. Thus, it is expected that when they are used with vaccines against not only Mycobacterium tuberculosis but also other pathogens, they remarkably increase the effectiveness of the existing vaccine for preventing infection, thereby being capable of effectively reducing the infection of various pathogens.

### TECHNICAL FIELD

The present invention relates to an immunological adjuvant and vaccine composition including STING agonist and a method for preventing infectious diseases using the same.

Further, the present invention relates to a vaccine composition including a STING agonist and an immunological adjuvant and a method for preventing infectious diseases using the same.

### BACKGROUND ART

Various strategies have been used in developing vaccines. Vaccines generated from live or attenuated bacteria are very effective, but safety problems have been often reported due to problems such as the risk of regression and sometimes self-replicating. It is known that although a vaccine using dead bacteria is effective for treatment, there is a problem that toxic substances such as LPS may be included and a possibility that living bacteria may exist (Ryan EJ et al., 2001). DNA vaccines developed to address these limitations are also inserted on the host's genome, increasing the mutagenic incidence or emerging potential of the oncogenic gene. On the other hand, subunit vaccines may be more stable than other vaccines since they use purified antigens. However, since these purified antigens often lack immunogenicity, a strong immunological adjuvant is required to increase such immunogenicity.

Meanwhile, c-di-GMP (cyclic diguanylate) was first identified as an intracellular signaling material that regulates cellulose production in Acetobacter xylinum. The signaling through c-di-GMP has been reported to specifically exist only in bacteria, not in eukaryotes. The role of c-di-GMP in bacteria is also reported as a second messenger of signaling. In particular, it is known that it regulates physiological mechanisms, which are important for the survival of bacteria, such as bacterial motility, adhesion, and cell-to-cell communication biofilm formation, and extracellular polysaccharide synthesis. Importantly, c-di-GMP derived from these bacteria acts as a ligand for the stimulator of IFN gene (STING), a cytosolic sensor of the host to induce production of Type I IFN through TBK1-IRF3 and production of NF-κB-mediated cytokines. Recently, it has been reported that STING agonists such as c-di-GMP may enhance antigen-specific T cells and humoral immune responses.

Therefore, the present inventors conducted the following experiments to confirm whether the STING agonist can be used as an immunological adjuvant in a vaccine.

### DISCLOSURE

### Technical Problem

The present invention is conceived to address the issues of the prior art as described above, and the object of the present invention is to provide an immunological adjuvant composition including a STING agonist as an active ingredient and a vaccine composition further including an antigen in the composition.

However, the technical problem to be achieved by the present invention is not limited to the problems mentioned above, and other problems that are not mentioned will be clearly understood by those of ordinary skill in the art from the following description.

### Technical Solution

The present invention provides an immunological adjuvant composition including a STING (stimulator of interferon genes) agonist as an active ingredient.

Further, the present invention provides a method of preventing infectious diseases including administering a STING agonist and an antigen to an individual.

### Advantageous Effects

It is confirmed that the immunological adjuvant composition and vaccine composition including the STING agonist according to the present invention not only may effectively activate various body immune responses but also have the effect of remarkably reducing the infection of Mycobacterium tuberculosis. Thus, it is expected that when they are used with vaccines against not only Mycobacterium tuberculosis but also other pathogens, they remarkably increase the effectiveness of the existing vaccine for preventing infection, thereby being capable of effectively reducing the infection of various pathogens.

### DESCRIPTION OF DRAWINGS

FIG. 1A is a view showing an experimental design for verifying the efficacy of c-di-GMP as an immunological adjuvant, and FIG. 1B is a view showing the results of confirming IFN-γ productivity after stimulation of ESAT-6 protein on the spleen cells of mice immunized with MPL or c-di-GMP based on ESAT-6 antigen. FIG. 1C is a view showing the results of confirming the generation of an ESAT-6 antigen-specific antibody in immunized mice, and FIG. 1D is a view showing the results of analyzing memory T cells infiltrated into the spleen and lung tissue of immunized mice.

FIG. 2A is a view showing the analysis method of multifunctional T cells, and FIG. 2B is a view showing the proportion of antigen-specific multifunctional T cells induced by stimulation of ESAT-6 protein in the lung and spleen cells of mice immunized with MPL or c-di-GMP based on ESAT-6 antigen.

FIGS. 3A and 3B are views showing the results of histopathological analysis of lung tissue 16 weeks after infection with Mycobacterium tuberculosis in mice immunized with MPL or c-di-GMP based on ESAT-6 antigen, and FIG. 3C is a view showing the results of measuring the number of Mycobacterium tuberculosis bacteria in the lungs and spleen.

FIG. 4A is a view showing an experimental design for confirming the synergistic effect of c-di-GMP and a conventional MPL immunological adjuvant, and FIG. 4B is a view showing the results of analyzing the activity of T cells in immunized mice. FIG. 4C is a view showing the results of confirming the generation of ESAT-6 antigen-specific antibodies in immunized mice by time, and FIG. 4D is a view showing results of analyzing the ratio of germinal center-related T cells and B cells in immunized mice.

FIG. 5A is a view showing the results of histopathological analysis of lung tissue four weeks after infection with Mycobacterium tuberculosis in mice immunized with MPL or c-di-GMP/MPL based on ESAT-6 antigen, and FIG. 5B is a view showing the results of measuring the number of Mycobacterium tuberculosis proliferated in the lungs. FIG. 5C is a view showing the results of the analysis of antigen-specific multifunctional T cells through ESAT-6 protein stimulation in cells separated from lungs and spleens ex vivo four weeks after infection with Mycobacterium tuberculosis in the completely immunized mice.

FIG. 6 is a view showing the ratio of antigen-specific multifunctional T cells induced by ESAT-6 protein stimulation in lung and spleen cells of mice immunized with c-di-GMP and/or GLA-SE based on ESAT-6 antigen.

FIG. 7 is a view showing the results of analyzing the activity of T cells in immunized mice.

FIG. 8 is a view showing the results of measuring the number of Mycobacterium tuberculosis in the lungs and spleen of mice immunized with GLA-SE alone or c-di-GMP and GLA-SE based on ESAT-6 antigen.

### BEST MODES OF THE INVENTION

Hereinafter, various embodiments described in the present application are described with reference to the drawings. In the following description, various specific details, such as specific forms, compositions, and processes are described for a thorough understanding of the present invention. However, certain embodiments may be implemented without one or more of these specific details, or in combination with other known methods and forms. In other examples, the known processes and manufacturing techniques have not been described in specific details in order not to unnecessarily obscure the present invention. Reference throughout this specification to “one embodiment” or “an embodiment” means that particular features, forms, compositions, or properties described in connection with the embodiment are included in one or more embodiments of the present invention. Thus, the context of “in one embodiment” or “an embodiment” expressed in various locations throughout this specification does not necessarily represent the same embodiment of the present invention. Further, particular features, forms, compositions, or properties may be combined in one or more embodiments in any suitable manner.

Unless otherwise defined in the specification, all scientific and technical terms used in the specification have the same meaning as commonly understood by one of ordinary skill in the art to which the present invention belongs.

The present invention provides an immunological adjuvant composition including a STING agonist as an active ingredient and a vaccine composition including a STING agonist and an antigen as active ingredients.

In one embodiment of the present invention, the STING agonist is preferably DNA, RNA, protein, peptide fragment, compound, etc. capable of activating STING signaling, more preferably c-di-GMP (cyclic diguanylate), cGAMP, 3′3′-cGAMP, c-di-GAMP, c-di-AMP, 2′3′-cGAMP, 10-(carboxymethyl)9(10H)acridone (CMA), 5,6-Dimethylxanthenone-4-acetic acid (DMXAA), methoxyvone, 6,4′-dimethoxyflavone, 4′-methoxyflavone, 3′,6′ -dihydroxyflavone, 7,2′ -dihydroxyflavone, daidzein, formononetin, retusin 7-methyl ether and xanthone, but is not limited thereto as long as it is a substance that may activate signaling by binding to STING.

In another embodiment of the present invention, the immunological adjuvant composition may additionally include other known immunological adjuvants, and other immunological adjuvants may preferably include at least one of monophosphoryl lipid A (MPL) and glucopyranosyl lipid immunological adjuvant, formulated in a stable nano-emulsion of squalene oil-in-water (GLA-SE)

In another embodiment of the present invention, the immunological adjuvant may be preferably encapsulated in a liposome, but is not limited thereto as long as it is in a form which is easy to inject into the body.

In another embodiment of the present invention, the antigen is a pathogen-specific antigen, preferably a Mycobacterium tuberculosis-specific antigen, more preferably an ESAT-6 antigen, or a conventional antigen that has been used in a subunit vaccine.

In another embodiment of the present invention, the vaccine composition is characterized in that it prevents infection of Mycobacterium tuberculosis.

The composition of the present invention may include one or more known active ingredients having an effect on preventing pathogens.

Further, the composition of the present invention may be prepared by including one or more pharmaceutically acceptable carriers for administration in addition to the above-described active ingredients. The pharmaceutically acceptable carriers may be used as saline, sterile water, Ringer's solution, buffered saline, dextrose solution, sucrose solution, glycerol, ethanol, and the mixture of one or more of these ingredients, and if necessary, other conventional additives such as antioxidants, buffers, bacteriostatic agents may be added. Further, diluents, dispersants, surfactants, binders, and lubricants may be further added to prepare injectable formulations such as aqueous solutions, suspensions, emulsions, pills, capsules, granules, or tablets. Further, it may be preferably formulated according to each disease or component by an appropriate method in the art or by using a method described in Remington's Pharmaceutical Science (latest edition), Mack Publishing Company, Easton, Pa.

The composition of the present invention may be orally or parenterally administered (for example, intravenous, subcutaneous, intraperitoneal, intramuscular, or topical application) according to a desired method, and the dosage range varies depending on the individual's weight, age, sex, health status, diet, administration time, administration method, excretion rate, the severity of disease and so on.

Further, the present invention provides a method for preventing infectious diseases, such as Mycobacterium tuberculosis infection, the method including administering a STING agonist and an antigen to an individual.

Further, the present invention provides a method for preventing infectious diseases, such as Mycobacterium tuberculosis infection, the method further including additionally administering at least one immunological adjuvant of monophosphoryl lipid A (MPL) and glucopyranosyl lipid immunological adjuvant, formulated in a stable nano-emulsion of squalene oil-in-water (GLA-SE).

The immunological adjuvant and the STING agonist may be administered simultaneously or sequentially.

In addition, the immunological adjuvant and STING agent are administered so that the infection of various Mycobacterium tuberculosis may be effectively prevented, and in particular, the production of multifunctional T cells in the spleen and/or lung is significantly increased compared to the case of using the immunological adjuvant alone so that the effect of the existing immunological adjuvant may be remarkably increased. Thus, the infection of Mycobacterium tuberculosis, such as highly pathogenic Mycobacterium tuberculosis, preferably HN878 strain, may be more effectively prevented.

The individual is preferably a mammal including humans, and may be an individual with an infectious disease, such as a Mycobacterium tuberculosis infection, and in this case, the antigen may be a Mycobacterium tuberculosis antigen.

Further, the present invention may be treated in combination with a substance for preventing conventional infectious diseases, such as Mycobacterium tuberculosis, in addition to the STING agonist and antigen.

Further, the present invention provides a use of an immunological adjuvant of a STING agonist and uses of STING agonist and antigen for preventing infectious diseases, such as Mycobacterium tuberculosis.

Further, the present invention provides a use of preparing an immunological adjuvant of a STING agonist and use of preparing a vaccine of a STING agonist and an antigen.

### Modes of the Invention

Hereinafter, the present invention will be described in more detail through Examples. These Examples are merely for describing the present invention in more detail. It is apparent to those of ordinary skill in the art that the scope of the present invention is not limited by these Examples according to the gist of the present invention.

### EXAMPLES

### Example 1: Confirmation of Role of STING Agonist as Immunological Adjuvant

The following experiment was conducted to confirm whether the STING agonist may be used as an immunological adjuvant against Mycobacterium tuberculosis.

### 1.1. Experimental Animals

The experiment was conducted by using 6-week-old female C57BL/6 mice without specific pathogens (Japan SLC, Inc. Shizuoka, Japan), and the mice were bred with sterilized commercial mouse feed and water in the limited space of ABSL-3 Biohazard Animal Room in Clinical Medicine Research Center, Yonsei University.

### 1.2. Experiment Design

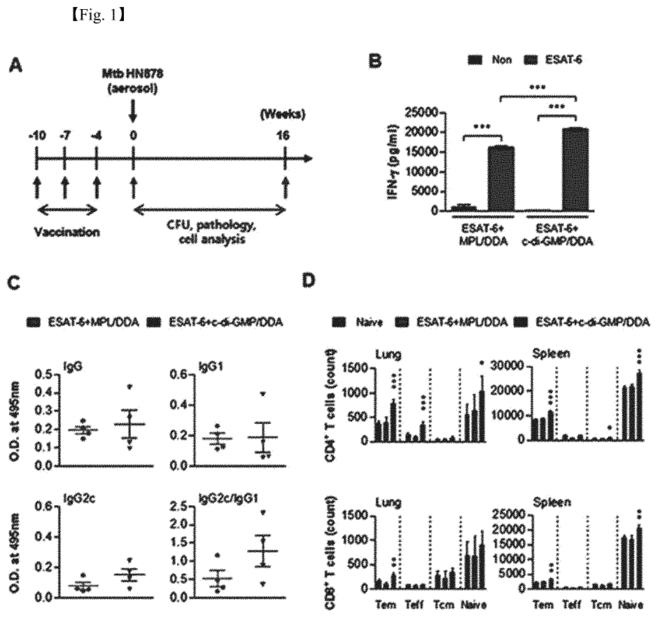

Animal experiments were conducted in order to confirm whether c-di-GMP (invivoGen), a type of STING (stimulator of interferon genes) agonist, may be used as an immunological adjuvant alone. A specific experimental method is shown in FIG. 1A, and monophosphoryl lipid A (MPL), which is a TLR4 agonist, which has been conventionally used as an immunological adjuvant, was used as a comparative control for c-di-GMP. Each immunological adjuvant was formulated with dimethyldioctadecylammonium (DDA) liposome in combination with or without ESAT-6 protein.

As shown in FIG. 1A, mice were immunized by three times intramuscular injection of MPL/DDA, ESAT-6+MPL/DDA or ESAT-6+c-di-GMP/DDA at three weeks intervals ten weeks before the mice were infected with Mycobacterium tuberculosis. Some mice were euthanized before infecting mice with Mycobacterium tuberculosis, and then spleen cells and lung cells were separated from the spleen and lungs. Then, ex vivo experiments were also performed. Each isolated cell was passaged and cultured in a general manner. The immunization method is described in detail in Example 1.3 below. Then, it was confirmed that the IFN-γ productivity by antigen stimulation and ESAT-6 protein immunization induced the increase of memory T cells that infiltrate into the spleen and lungs. Further, the ability to form multifunctional CD4+ T cells, which is recently reported to be important for tuberculosis defense, was examined. The number of bacteria in the spleen and lungs and the degree of inflammation in the lungs were analyzed at the 16th week after Mycobacterium tuberculosis infection.

### 1.3. Immunization

For immunization, each mouse used in the experiment was intramuscularly injected with the experimental vaccine composition on its back three times at three weeks intervals. The experimental vaccine composition was diluted to a final concentration of 5 mg/mL of DDA liposome, then heated to 65° C., and dissolved in combination with ultrasonic sonication, and then mixed with each immunological adjuvant to a volume of 2:1 to be prepared. The volume injected per mouse was set to be 200 μl, and 1 μg of ESAT-6 protein per injection was included as an antigen. As a negative control, an MPL/DDA composition without an antigen was injected intramuscularly.

### 1.4. Confirmation of IFN-γ Productivity by ESAT-6 Antigen

In each experimental mouse group, the antigen-specific IFN-γ productivity was analyzed in order to verify the efficacy of whether c-di-GMP/DDL immunological adjuvants affect the immunogenicity of immunized antigens compared with the MPL/DDA immunological adjuvant which was conventionally used as an immunological adjuvant. For the experiment, mouse spleen cells obtained in the same manner as in Example 1.2 were stimulated ex vivo using ESAT-6 protein. The antigen-specific IFN-γ productivity in the stimulated cells was confirmed by enzyme-linked immunosorbent assay (ELISA). The results are shown in FIG. 1B.

A shown in FIG. 1B, it was confirmed that antigen-specific IFN-γ was produced in both the experimental groups immunized using the ESAT-6+MPL/DDA vaccine composition and the ESAT-6+c-di-GMP/DDA vaccine composition against ESAT-6 protein stimulation. It was confirmed that the IFN-γ productivity in the experimental group immunized with the ESAT-6+c-di-GMP/DDA vaccine composition using c-di-GMP as an immunological adjuvant among them was significantly increased compared with the conventional immunological adjuvant.

### 1.5. Confirmation of Antigen-Specific Antibody Productivity

In order to verify the efficacy of whether the c-di-GMP/DDL immunological adjuvant has an effect on the antigen-specific antibody productivity, serum of each mouse was separated at the time when immunization was finally completed over a total of 3 times, so as to confirm the degree of antibody production. ESAT-6 protein was added at a concentration of 1 μg/ml to a 96-well plate and then was reacted at room temperature for 2 hours so as to coat each well. Then, mouse serum according to each experimental group was added to the coated wells. Further, after anti-IgG (Sigma), anti-IgG1 (BD Bioscience), or anti-IgG2c (Southern Biotech) to which horseradish peroxidase (HRP) is bound was added to each well to react, the generated antigen-specific antibody productivity was confirmed using ELISA. The results are shown in FIG. 1C.

As shown in FIG. 1C, it was confirmed that both IgG1 related to Th2 immunity as well as total IgG and IgG2c related to Th1 immunity, which is reported to be important for the defense of tuberculosis, have improved antibody productivity than the conventional immunological adjuvant.

### 1.6. Confirmation of Type and Number of T Cells Induced When c-di-GMP is Used as Immunological Adjuvant

In general, when infected with Mycobacterium tuberculosis, Mycobacterium tuberculosis antigen-specific T cells replicate and differentiate to become effector T cells. When the immune response disappears, most of these effector T cells die, and some form long-lasting memory T cells. It is known that the memory T cells thus formed play a critical role in the formation of a rapid acquired defense immunity and maintenance of the memorization of T cells. Therefore, an experiment was conducted to verify whether immunization with ESAT-6 protein using MPL or c-di-GMP as an immunological adjuvant has an effect on memory T cells. For the experiment, the spleen and lung cells of the mouse obtained in the same manner as in Example 1.2 were washed twice with PBS and then centrifuged. The centrifuged cells were treated with anti-CD3-BV421 (BD Bioscience), anti-CD4-PerCP-Cy5.5 (BD Bioscience), anti-CD8-APC-Cy7 (BD Bioscience), anti-CD44-PE (Ebioscience), anti-CD62L-FITC (Ebioscience), anti-CD127-APC (Ebioscience), etc. Then, they were reacted at 4° C. for 30 minutes. After the reaction was completed, cells were washed several times with PBS to remove unbound antibody, and then effector T cells (Teff; CD3⁺CD4⁺CD62L⁻CD44+CD127⁻), effector/memory T cells (Tem; CD3⁺CD4⁺CD62L⁻CD44⁺CD127⁺), central memory T cells (Tcm; CD3⁺CD4⁺CD62L⁺CD44⁺CD127⁺), and naive T cells (Naive; CD3⁺CD4⁺CD62L⁺CD44⁻CD127⁺) were analyzed by using flow cytometer (FACS verse, BD). The results are shown in FIG. 1D.

As shown in FIG. 1D, it was confirmed that the immunization used c-di-GMP as an immunological adjuvant has a significant increase of effector/memory T of CD4⁺/CD8⁺ in both spleen and lung cells of mice compared with the use of MPL as an immunological adjuvant.

### 1.7. Confirmation of T Cell Activity Induced by Using c-di-GMP Immunological Adjuvant

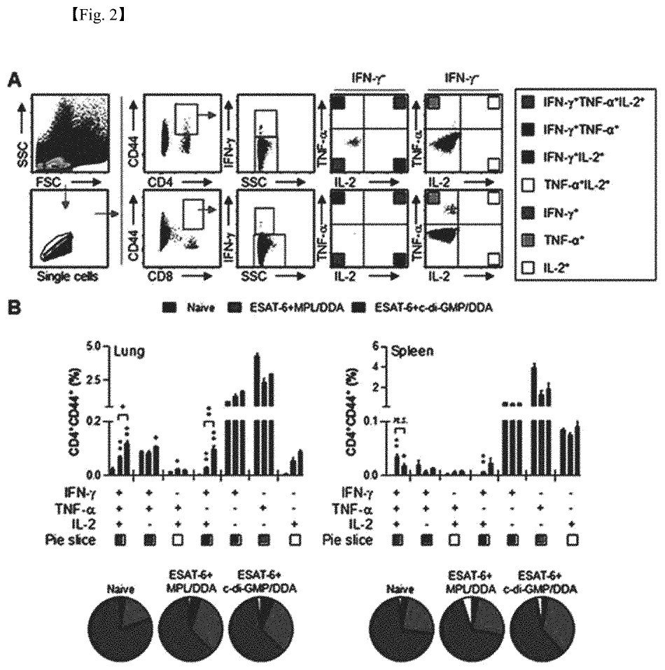

Since multifunctional T cells (T cells that secrete both IFN-γ, TNF-α and/or IL-2) are reported as an essential immunological indicator to defend against Mycobacterium tuberculosis, it was confirmed whether multifunctional T cells were generated when c-di-GMP was used as an immunological adjuvant. The spleen and lung cells of the mouse isolated in the same manner as in Example 1.2 were stimulated using ESAT-6 protein. Further, the cells were treated with GolgiStop (BD Bioscience) to prevent the generated cytokines from being secreted to the outside of the cells. Then, the cells were reacted at 37° C. for 12 hours, and then washed twice with PBS. The washed cells were treated with anti-CD4-PerCP-Cy5.5 (BD Bioscience), anti-CD8-APC-Cy7 (BD Bioscience), anti-CD44-v450 (BD Bioscience), etc. Then, they were reacted at 4° C. for 30 minutes. Unbound antibody was removed by washing with PBS. Further, in order to increase the permeability of the cells, Cytofix/Cytoperm (BD Bioscience) was added to the cells, reacted at 4° C. for 30 minutes, and then washed again with PBS, and the cells were additionally treated with anti-IFN--65 PE (BD Bioscience), anti-IL-2-PE-Cy7 (BD Bioscience), anti-TNF-α-APC (BD Bioscience), and the like, and were reacted at 4° C. for 30 minutes. The cells in which the reaction was completed were washed several times with Perm/Wash solution (BD Bioscience) and analyzed by flow cytometer, and the results are shown in FIG. 2A. Further, the results analyzed through the flow cytometer were further analyzed with a gating strategy using FlowJo software, and the results are shown in FIG. 2B.

As shown in FIG. 2B, it was confirmed that T cells secreting all three types of cytokines IFN-γ, TNF-α and IL-2 were increased in all cases where c-di-GMP or MPL was used as an immunological adjuvant. In particular, it was confirmed that in lung cells, the generation of antigen-specific multifunctional T cells in the group immunized with the ESAT-6+c-di-GMP/DDA composition was significantly increased than the case in which MPL was used as an immunological adjuvant. These confirmed that compared with conventional MPL, STING agonists increase the production of multifunctional T cells, which are essential for the prevention and treatment of Mycobacterium tuberculosis so that they may be effectively used than conventional immunological adjuvants for the vaccine of the prevention and treatment of Mycobacterium tuberculosis.

### 1.8. Mycobacterium tuberculosis Culture and Air Infection

The highly pathogenic Mycobacterium tuberculosis HN878 strain was cultured in 7H9-OADC broth for 15 days. Then, after collecting the cultured bacteria, the bacteria were crushed by gentle vortexing with 6 mm glass beads. The crushed bacteria were centrifuged to settle the cell aggregates and then collect the supernatant. The collected supernatant was divided and dispensed, and they were stored at −70° C. The stored bacteria were thawed at the time of the experiment, diluted sequentially in Middlebrook 7H11 Agar (Difco, Detroit, Mich., USA), and cultured to check the number of bacteria. In order to infect mice, Mycobacterium tuberculosis was gently sonicated in a sonic bath and then diluted with PBS (pH 7.2) to obtain a desired number of Mycobacterium tuberculosis. The infection of Mycobacterium tuberculosis was performed by the air infection through inhalation of 200 to 250 Mycobacterium tuberculosis per mouse using a Glas-Col inhalation device (Terre Haute, Ind.), which is an air-infection device.

### 1.9. Histopathological Analysis and Confirmation of Inhibitory Effect of Mycobacterium tuberculosis Infection

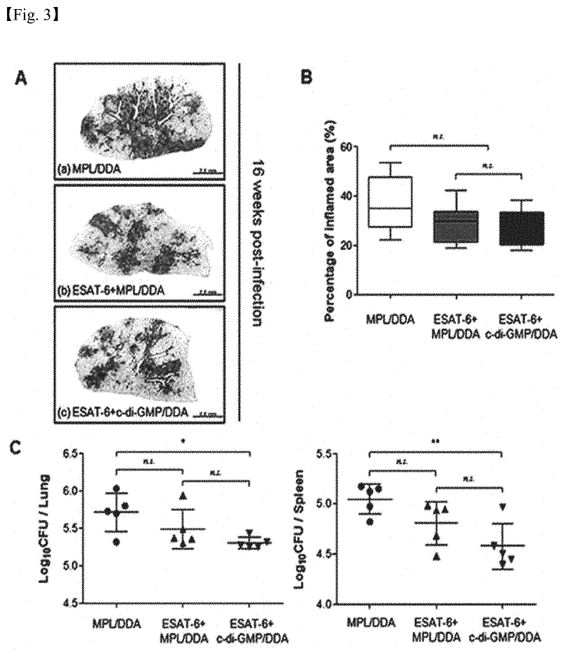

To confirm whether the immunization reaction using c-di-GMP as an immunological adjuvant has an effect of inhibiting Mycobacterium tuberculosis infection, the mice were infected with Mycobacterium tuberculosis in the same manner as in Example 1.8. The infected mice were euthanized after 16 weeks, and the lung tissue was removed from each of the mice of the experimental group. Further, after the lung tissue was preserved in 10% neutral-buffered formalin, it was fixed in paraffin. The fixed lung tissue was sectioned with a thickness of 4 to 5 mm and stained with Hematoxylin & Eosin (H&E). The results are shown in FIG. 3A. By using the stained result, the area of the area where inflammation appeared was quantified using the ImageJ software program (National Institute of Health, MD, USA). The results are shown in FIG. 3B. Further, Mycobacterium tuberculosis bound to the lungs and spleens of euthanized mice were removed with PBS to obtain a homogenous suspension. Each homogenous suspension was diluted step by step, and then cultured in Middlebrook 7H11 Agar (Difco, Detroit, Mich., USA). Thus, the number of the infected Mycobacterium tuberculosis was confirmed, and the number of Mycobacterium tuberculosis infected was expressed as an average log₁₀ CFU±standard deviation per total lung or spleen tissue. The results are shown in FIG. 3C.

As shown in FIG. 3B, in the lung tissue of the mouse immunized with an immunological adjuvant, inflammation was reduced compared to the negative control using only an immunological adjuvant. In particular, it can be confirmed that the experimental group immunized using the ESAT-6+c-di-GMP/DDA vaccine composition had less inflammation than that in the experimental group immunized using the ESAT-6+MPL/DDA vaccine composition.

Further, as shown in FIG. 3C, it can be confirmed that the number of infected Mycobacterium tuberculosis bacteria was significantly reduced in the experimental group immunized using the ESAT-6+c-di-GMP/DDA vaccine composition compared to the negative control group immunized using only the immunological adjuvant. These confirm that c-di-GMP alone as an immunological adjuvant is used to effectively prevent the infection of the highly pathogenic Mycobacterium tuberculosis HN878 strain.

Through the above results, it can be confirmed that a STING agonist such as c-di-GMP (cyclic diguanylate) alone has immunological adjuvanticity, and its effect is enhanced compared to MPL, which is a conventional immunological adjuvant. Further, it can be confirmed that the STING agonist alone may be used as an immunological adjuvant to effectively prevent infection of various Mycobacterium tuberculosis.

### Example 2: Confirmation of Synergistic Effect of STING Agonist and Immunological Adjuvant

The following experiment was conducted to confirm the synergistic effect in the case of using the stimulator of interferon genes (STING) agonist in combination with MPL, a known immunological adjuvant.

### 2.1. Experimental Animals

The experiment was conducted by using 6-week-old female C57BL/6 mice without specific pathogens (Japan SLC, Inc. Shizuoka, Japan), and the mice were bred with sterilized commercial mouse feed and water in the limited space of ABSL-3 Biohazard Animal Room in Clinical Medicine Research Center, Yonsei University.

### 2.2. Experiment Design

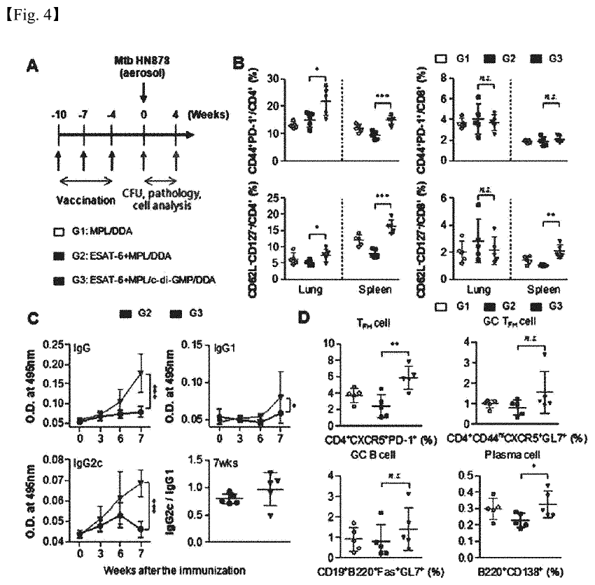

Animal experiments were conducted to confirm whether synergistic effects appeared when c-di-GMP (invivoGen) and conventionally known immunological adjuvant were used in combination. A specific experimental method is shown in FIG. 4A. c-di-GMP and MPL were formulated together in DDA liposomes, and they were used in the experimental group.

As shown in FIG. 4A, mice were immunized by three times intramuscular injection of vaccine compositions at three weeks intervals ten weeks before the mice were infected with Mycobacterium tuberculosis. Some mice were euthanized before infecting mice with Mycobacterium tuberculosis, and then spleen cells and lung cells were separated from the spleen and lungs. Then, ex vivo experiments were also performed. Each isolated cell was passaged and cultured in a general manner. The immunization method is described in detail in Example 2.3 below. Then, it was confirmed that the IFN-γ productivity by antigen stimulation and ESAT-6 protein immunization induced the increase of memory T cells that infiltrate into the spleen and lungs. Further, the ability to form multifunctional CD4+ T cells, which is recently reported to be important for tuberculosis defense, was examined. The number of bacteria in the spleen and lungs and the degree of inflammation in the lungs were analyzed at the 4th week after Mycobacterium tuberculosis infection.

### 2.3. Immunization

For immunization, each mouse used in the experiment was intramuscularly injected with the experimental vaccine composition on its back three times at three weeks intervals. The experimental vaccine composition was diluted to a final concentration of 5 mg/mL of DDA liposome, then heated to 65° C., and dissolved in combination with ultrasonic sonication, and then mixed to be a volume of 2:1. The volume injected per mouse was set to be 200 μl, and 1 μg of ESAT-6 protein per injection was included as an antigen. As a negative control, an MPL/DDA composition without an antigen was injected intramuscularly.

### 2.4. Confirmation of Activity of CD4⁺/CD8⁺ T Cells

In order to confirm whether the complex immunological adjuvant affects the activity of T cells, the T cell activity markers CD44, PD-1, CD62L, and CD127 were used to confirm the activity of T cells in the same manner as in Example 1.11. The results are shown in FIG. 4B.

As shown in FIG. 4B, it was confirmed that the case in which c-di-GMP and MPL were used in combination had the increased number of CD8⁺ T cells in the spleen and the increased number of CD44⁺PD-1⁺ and CD62L⁻CD127⁻ T cells in both the spleen and lungs for CD4⁺ T cells compared to the case of using a single immunological adjuvant.

### 2.5. Confirmation of Antigen-Specific Antibody Productivity

To verify the efficacy of whether the complex immunological adjuvant has an effect on the antigen-specific antibody productivity, serum of each mouse was separated at the time when immunization was finally completed over a total of three times, so as to confirm the degree of antibody production. ESAT-6 protein was added at a concentration of 1 μg/ml to a 96-well plate and then was reacted at room temperature for 2 hours so as to coat each well. Then, mouse serum according to each experimental group was added to the coated wells. Further, after anti-IgG (Sigma), anti-IgG1 (BD Bioscience), or anti-IgG2c (Southern Biotech) to which horseradish peroxidase (HRP) is bound was added to each well to react, the generated antigen-specific antibody productivity was confirmed using ELISA. The results are shown in FIG. 4C.

As shown in FIG. 4C, it was confirmed that in both IgG1 related to Th2 immunity as well as total IgG and IgG2c related to Th1 immunity, which is reported to be important for the defense of tuberculosis, the complex immunological adjuvant with c-di-GMP and MPL have improved antibody productivity than the immunological adjuvant alone. These results confirmed that a balanced immune response was induced.

### 2.6. Identification of Germinal Center-Related Cells

It is known that the germinal center (GC) is located in the follicle of B cells in the secondary lymphoid tissue, and the B cells related to the germinal center undergoes an important functional maturation process in regulating protective humoral immunity such as somatic hypermutation and memory formation of B cell. Further, it is reported follicular helper T cells (T_{FH}), a type of CD4⁺ T cell, are important cells that can enhance the antibody response of B cells, and the expression of the CXCR5 gene in T_{FH} cells plays an important role in causing T_{FH} cells to migrate to the B cell region and interacting with cognate B cells. Thus, it is important to enhance the activity of T_{FH} cells, which may induce an immune response of the germinal center, in order for the constant fixation of an effector with high affinity through humoral immunity. Therefore, in order to confirm whether the complex immunological adjuvant of the present invention activates T_{FH} cells, spleen cells were isolated from the finally immunized mouse, and germinal center-related cells were identified using a flow cytometer. The results are shown in FIG. 4D.

As shown in FIG. 4D, it was confirmed that the number of CD4⁺CXCR5⁺PD-1⁺ T_{FH} cells and B220⁺CD138⁺plasma cells was significantly increased in the experimental group using the complex immunological adjuvant of c-di-GMP and MPL, compared to the experimental group using MPL alone as an immunological adjuvant. This seems to have been affected by mediation of STING signaling caused by c-di-GMP. Meanwhile, it was confirmed that it increased slightly, but there was no significant difference in the case of the germinal center-related cells, CD4⁺CD44^{hi}CXCR5⁺GL7⁺ T_{FH} cells (GC T_{FH} cell) and CD19⁺B220⁺Fas⁺GL7⁺ B cells (GC B cell).

### 2.7. Mycobacterium tuberculosis Culture and Air Infection

The highly pathogenic Mycobacterium tuberculosis HN878 strain was cultured in 7H9-OADC broth for 15 days. Then, after collecting the cultured bacteria, the bacteria were crushed by gentle vortexing with 6 mm glass beads. The crushed bacteria were centrifuged to settle the cell aggregates and then collect the supernatant. The collected supernatant was divided and dispensed, and they were stored at −70° C. The stored bacteria were thawed at the time of the experiment, diluted sequentially in Middlebrook 7H11 Agar (Difco, Detroit, Mich., USA), and cultured to check the number of bacteria. In order to infect mice, Mycobacterium tuberculosis was gently sonicated in a sonic bath and then diluted with PBS (pH 7.2) to obtain a desired number of Mycobacterium tuberculosis. The infection of Mycobacterium tuberculosis was performed by the air infection through inhalation of 200 to 250 Mycobacterium tuberculosis per mouse using a Glas-Col inhalation device (Terre Haute, Ind.), which is an air-infection device.

### 2.8. Histopathological Analysis and Confirmation of Inhibitory Effect of Mycobacterium tuberculosis Infection

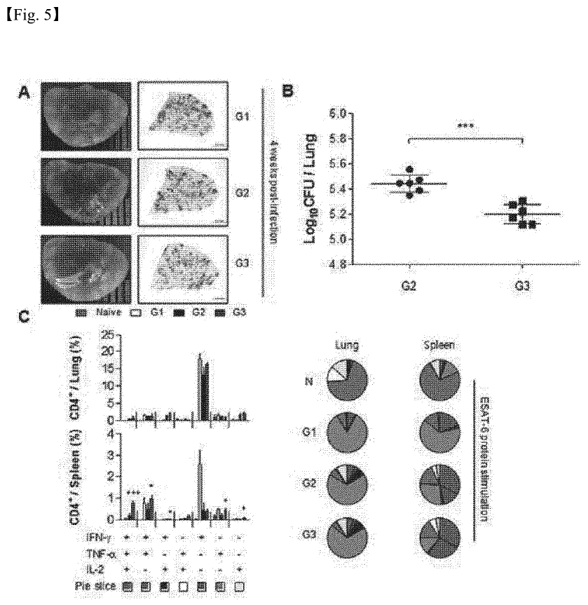

To confirm whether the immunization reaction using c-di-GMP as an immunological adjuvant has an effect of inhibiting Mycobacterium tuberculosis infection, the mice were infected with Mycobacterium tuberculosis in the same manner as in Example 2.7. The infected mice were euthanized after four weeks, and the lung tissue was removed from each of the mice of the experimental group. Further, after the lung tissue was preserved in 10% neutral-buffered formalin, it was fixed in paraffin. The fixed lung tissue was sectioned with a thickness of 4 to 5 mm and stained with Hematoxylin & Eosin (H&E). The results are shown in FIG. 5A. Further, Mycobacterium tuberculosis bound to the lungs of euthanized mice were removed with PBS to obtain a homogenous suspension. Each homogenous suspension was diluted step by step, and then cultured in Middlebrook 7H11 Agar (Difco, Detroit, Mich., USA). Thus, the number of the infected Mycobacterium tuberculosis was confirmed, and the number of Mycobacterium tuberculosis infected was expressed as an average log₁₀ CFU±standard deviation per total lung or spleen tissue. The results are shown in FIG. 5B.

As shown in FIG. 5A, it was confirmed that the inflammation in the lung tissue of mice immunized using a complex immunological adjuvant was reduced compared to the experimental group using only the MPL immunological adjuvant.

Further, as shown in FIG. 5B, it was confirmed that the number of infected Mycobacterium tuberculosis in the experimental group immunized using the complex immunological adjuvant was significantly reduced compared to the experimental group using only the MPL immunological adjuvant. It was confirmed that the case in which c-di-GMP and MPL are used in combination as an immunological adjuvant, may more effectively prevent infection of the highly pathogenic Mycobacterium tuberculosis HN878 strain than when used alone.

### 2.9. Confirmation of T Cell Activity Induced When Using Complex Immunological Adjuvant

Since multifunctional T cells (T cells that secrete both IFN-γ, TNF-α and/or IL-2) are reported as an essential immunological indicator to defend against Mycobacterium tuberculosis, it was confirmed whether multifunctional T cells were generated when complex immunological adjuvant was used. After infecting the immunized mice with Mycobacterium tuberculosis, the spleen and lung cells of the mouse isolated in the same manner as in Example 2.2 were stimulated using ESAT-6 protein. Further, the cells were treated with GolgiStop (BD Bioscience) to prevent the generated cytokines from being secreted to the outside of the cells. Then, the cells were reacted at 37° C. for 12 hours, and then washed twice with PBS. The washed cells were treated with anti-CD4-PerCP-Cy5.5 (BD Bioscience), anti-CD8-APC-Cy7 (BD Bioscience), anti-CD44-v450 (BD Bioscience), etc. Then, they were reacted at 4° C. for 30 minutes. Unbound antibody was removed by washing with PBS. Further, in order to increase the permeability of the cells, Cytofix/Cytoperm (BD Bioscience) was added to the cells, reacted at 4° C. for 30 minutes, and then washed again with PBS, and the washed cells were additionally treated with anti-IFN-γ-PE (BD Bioscience), anti-IL-2-PE-Cy7 (BD Bioscience), anti-TNF-α-APC (BD Bioscience), and the like, and were reacted at 4° C. for 30 minutes. The cells in which the reaction was completed were washed several times with Perm/Wash solution (BD Bioscience) and analyzed by flow cytometer. Further, the results analyzed were further analyzed with a gating strategy using FlowJo software, and the results are shown in FIG. 5C.

As shown in FIG. 5C, it was confirmed that the multifunctional T cells of CD4⁺IFN-γ⁺TNF-α⁺IL-2⁺, CD4⁺IFN-γ⁺TNF-α⁺ and CD4⁺IFN-γ⁺IL-2⁺ in the case in which c-di-GMP and MPL were used as an immunological adjuvant in combination were significantly increased compared to the experimental group using a single immunological adjuvant.

The above results may indicate that the effect was remarkably increased when using a conventional immunological adjuvant such as MPL in combination with a STING agonist such as c-di-GMP. Further, through this, it can be confirmed that the additional use of a STING agonist to the conventional immunological adjuvants could effectively prevent infection of various Mycobacterium tuberculosis.

### Example 3: Confirmation of Synergistic Effect of STING Agonist and Conventional Immunological Adjuvant

The following experiment was conducted to confirm the synergistic effect in the case of using the stimulator of interferon genes (STING) agonist in combination with glucopyranosyl lipid immunological adjuvant, formulated in a stable nano-emulsion of squalene oil-in-water (GLA-SE), a known immunological adjuvant.

### 3.1. Confirmation of T Cell Activity Induced When Using STING Agonist and GLA-SE

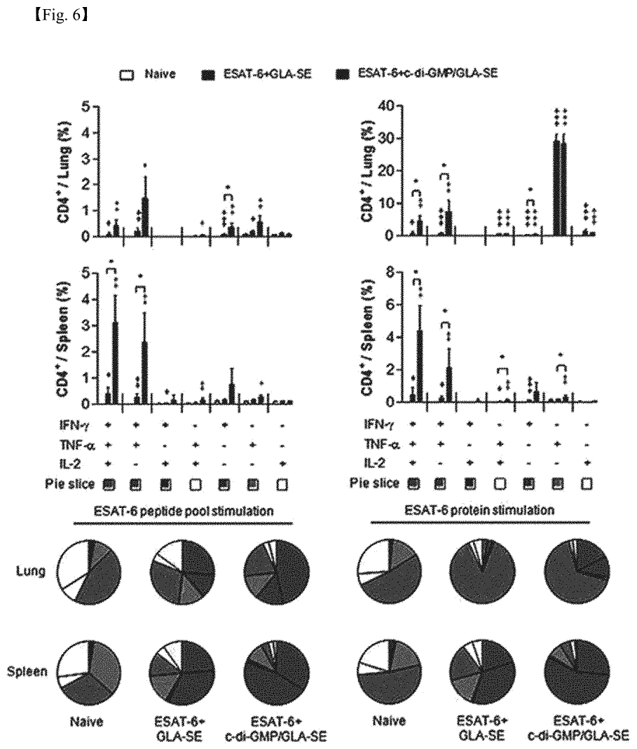

In order to confirm whether the complex immunological adjuvant affects the activity of T cells, the spleen and lungs were isolated before mice immunized with the single immunological adjuvant or the complex immunological adjuvant were infected with Mycobacterium tuberculosis. Further, the generation of antigens and T cells induced after treatment with the ESAT-6 peptide pool or protein in ex vivo was confirmed using a flow cytometer. The detailed experimental manner was performed in the same manner as in Example 1.7. The results are shown in FIG. 6.

As shown in FIG. 6, it was confirmed that in all experimental groups, the generation of multifunctional T cells of CD4⁺IFN-γ⁺TNF-α⁺ was significantly increased in the group immunized using the complex immunological adjuvant of c-di-GMP and GLA-SE compared to the group immunized with GLA-SE alone.

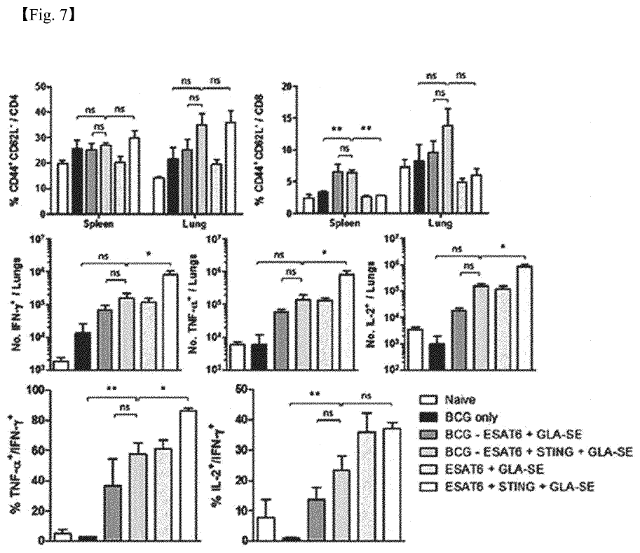

Further, in order to verify of efficiency for total 7 groups of (1) negative control group, (2) tuberculosis alone treatment group, (3) BCG (Bacille de Calmette-Guerin vaccine) alone treatment group, (4) BCG prime, ESAT-6, and GLA-SE treatment group, (5) BCG prime, ESAT-6, GLA-SE and STING agonist (c-di-GMP) treatment group, (6) ESAT-6 and GLA-SE treatment group, and (7) ESAT-6, STING agonist and GLA-SE treatment group, the mice were euthanized at the time when immunization for a total of 3 times was finally completed, and then the activity of T cells was confirmed in the same manner as in Examples 1.5, 1.7 and 1.11. the intramuscular injection was used for immunization. The results are shown in FIG. 7.

As shown in FIG. 7, it was confirmed that the secretion of cytokines such as IFN-γ, TNF-α, and IL-2 was increased in the case in which c-di-GMP and GLA-SE were used in combination, compared to the case of using a single complex immunological adjuvant.

Through the above results, it was confirmed that the use of GLA-SE and STING agonist as a complex immunological adjuvant could effectively prevent infection of various Mycobacterium tuberculosis.

### 3.2. Confirmation of Inhibitory Effect on Mycobacterium tuberculosis Infection When Using STING Agonist and GLA-SE

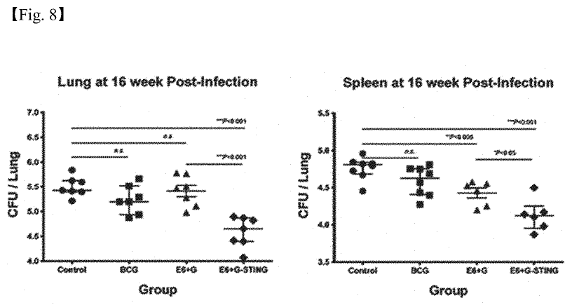

To confirm whether the immunization reaction by using the complex immunological adjuvant has an inhibitory effect on Mycobacterium tuberculosis infection, mice were immunized by three times intramuscular injection of vaccine compositions of (1) BCG (Bacille de Calmette-Guerin vaccine) alone, (2) ESAT-6 and GLA-SE, (3) ESAT-6, STING agonist and GLA-SE at three weeks intervals ten weeks before the mice were infected with Mycobacterium tuberculosis. Mice were infected in the same manner as in Example 2.7. After four weeks of infection with Mycobacterium tuberculosis, the infected mice were euthanized, and the Mycobacterium tuberculosis attached to the lungs of each mouse was extracted with PBS to obtain a homogenous suspension, and each homogenous suspension was diluted step by step, and then was cultured in Middlebrook 7H11 Agar (Difco, Detroit, Mich., USA). Thus, the number of the infected Mycobacterium tuberculosis was confirmed, and the number of Mycobacterium tuberculosis infected was expressed as an average log₁₀ CFU±standard deviation per total lung or spleen tissue. The results are shown in FIG. 8.

As shown in FIG. 8, it was confirmed that the case in which c-di-GMP and GLA-SE were used simultaneously as a complex immunological adjuvant, significantly reduced the number of infected Mycobacterium tuberculosis compared to the experimental group using GLA-SE alone as an immunological adjuvant. It was confirmed that the case in which c-di-GMP and GLA-SE are used in combination as an immunological adjuvant, may more effectively prevent infection of the highly pathogenic Mycobacterium tuberculosis HN878 strain than when used alone.

Through the above results, it can be confirmed that the effect was remarkably increased when the conventional immunological adjuvants such as MPL and GLA-SE were used in combination with a STING agonist such as c-di-GMP. In particular, it has been recently found that the generation of multifunctional T cells of CD4⁺IFN-γ⁺TNF-α⁺ is essential for the prevention and treatment of Mycobacterium tuberculosis, but it can be confirmed that the STING agonist of the present invention significantly increases the production of multifunctional T cells in the spleen and/or lung compared to the case of using other conventional immunological adjuvants alone. Through the above results, it can be confirmed that the addition of STING agonist to the conventionally used immunological adjuvant may increase the effect of the conventional immunological adjuvant having the low prevention and treatment effect against the existing Mycobacterium tuberculosis. Accordingly, it can be confirmed that a complex immunological adjuvant containing a STING agonist may effectively prevent infection of various Mycobacterium tuberculosis.

As described above, certain parts of the present invention have been described in detail, but it is clear that these specific techniques are only preferred embodiments and are not limited to the scope of the present invention for those of ordinary skill in the art. Therefore, the substantial scope of the present invention will be defined by the appended claims and their equivalents.

1. An immunological adjuvant composition comprising a stimulator of interferon gene (STING) agonist as an active ingredient.

2. The immunological adjuvant composition of claim 1, wherein the STING agonist includes at least one selected from the group consisting of c-di-GMP (cyclic diguanylate), cGAMP, 3′3′-cGAMP, c-di-GAMP, c-di-AMP, and 2′3′-cGAMP.

3. The immunological adjuvant composition of claim 1, wherein the STING agonist includes at least one selected from the group consisting of c-di-GMP (cyclic diguanylate), cGAMP, 3′3′-cGAMP, c-di-GAMP, c-di-AMP, 2′3′-cGAMP, 10-(carboxymethyl)9(10H)acridone (CMA), 5,6-Dimethylxanthenone-4-acetic acid (DMXAA), methoxyvone, 6,4′-dimethoxyflavone, 4′-methoxyflavone, 3′,6′-dihydroxyflavone, 7,2′-dihydroxyflavone, daidzein, formononetin, retusin 7-methyl ether and xanthone.

4. The immunological adjuvant composition of claim 1, wherein the composition further includes at least one immunological adjuvant of monophosphoryl lipid A (MPL) and glucopyranosyl lipid immunological adjuvant formulated in a stable nano-emulsion of squalene oil-in-water (GLA-SE).

5. The immunological adjuvant composition of claim 4, wherein the immunological adjuvant is encapsulated in a liposome.

6. A vaccine composition comprising a stimulator of interferon gene (STING) agonist and an antigen as active ingredients.

7. The vaccine composition of claim 6, wherein the STING agonist includes at least one selected from the group consisting of c-di-GMP (cyclic diguanylate), cGAMP, 3′3′-cGAMP, c-di-GAMP, c-di-AMP, and 2′3′-cGAMP.

8. The vaccine composition of claim 6, wherein the STING agonist includes at least one selected from the group consisting of c-di-GMP (cyclic diguanylate), cGAMP, 3′3′-cGAMP, c-di-GAMP, c-di-AMP, 2′3′-cGAMP, 10-(carboxymethyl)9(10H)acridone (CMA), 5,6-Dimethylxanthenone-4-acetic acid (DMXAA), methoxyvone, 6,4′-dimethoxyflavone, 4′-methoxyflavone, 3′,6′-dihydroxyflavone, 7,2′-dihydroxyflavone, daidzein, formononetin, retusin 7-methyl ether and xanthone.

9. The vaccine composition of claim 6, wherein the composition further includes at least one immunological adjuvant of monophosphoryl lipid A (MPL) and glucopyranosyl lipid immunological adjuvant, formulated in a stable nano-emulsion of squalene oil-in-water (GLA-SE).

10. The vaccine composition of claim 9, wherein the immunological adjuvant is encapsulated in a liposome.

11. The vaccine composition of claim 6, wherein the antigen is a Mycobacterium tuberculosis-specific antigen.

12. The vaccine composition of claim 11, wherein the Mycobacterium tuberculosis-specific antigen is ESAT-6.

13. The vaccine composition of claim 6, wherein the vaccine composition is to prevent infection of Mycobacterium tuberculosis.

14. A method of preventing an infectious disease, the method comprising administering a stimulator of interferon gene (STING) agonist and an antigen.

15. The method of claim 14, the method further comprising administering at least one immunological adjuvant of monophosphoryl lipid A (MPL) and glucopyranosyl lipid immunological adjuvant, formulated in a stable nano-emulsion of squalene oil-in-water (GLA-SE).

16. The method of claim 14, wherein the infectious disease is Mycobacterium tuberculosis.
